SEQUENCE LISTING (2 sequences)

SEQ ID NO: 1 - DNA, 6169 bp, Homo sapiens
  CDS (1)..(6045)
atggcaaacttcctattacctaggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggtcccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcamaaaccagcctggggccttcctggctttgggagtaagaaatgggcct

SEQ ID NO: 2 - protein, 2015 aa, Homo sapiens [sequence omitted]

(19) United States
(12) Patent Application Publication
(10) Pub. No.: US 2010/0112562 A1
(43) Pub. Date: May 6, 2010

(54) Mutation Implicated in Abnormality of Cardiac Sodium Channel Function

(75) Inventors: Yang Soo Jang, Seoul (KR); Sung Soon Kim, Seoul (KR); SungJoo Kim Yoon, Seoul (KR); Dong-Jik Shin, Seoul (KR)

Correspondence Address: THE NATH LAW GROUP, 112 South West Street, Alexandria, VA, 22314, US

(73) Assignees: INDUSTRY-ACADEMIC COOPERATION FOUNDATION YONSEI UNIVERSITY, Seoul (KR); INDUSTRY-ACADEMIC COOPERATION FOUNDATION THE CATHOLIC UNIVERSITY OF KOREA, Seoul (KR)

(21) Appl. No.: 12/329,540

(22) Filed: Dec. 5, 2008

Related U.S. Application Data
(60) Provisional application No. 61/023,423, filed on Jan. 24, 2008.

(51) Int. Cl. C12Q 1/68; C07H 21/04; C07K 14/00; G01N 33/53

(52) U.S. Cl. 435/6; 536/23.5; 530/350; 436/94

(57) ABSTRACT

A novel mutation in the SCN5A gene is associated with loss of cardiac sodium channel function. Analysis of the novel mutation provides an early diagnosis of subjects with cardiac diseases or disorders caused by loss of cardiac sodium channel function, particularly Brugada syndrome. Diagnostic methods include analyzing the sequences of the SCN5A gene or protein of an individual to be tested and comparing them with the sequences of the native, nonvariant SCN5A gene or protein. Pre-symptomatic diagnosis of these syndromes will enable practitioners to treat these disorders using existing medical therapy, e.g., using sodium channel blockers or through electrical stimulation.

### CROSS-REFERENCE TO RELATED APPLICATION

This is a non-provisional of U.S. Provision Application 61/023,423, filed on Jan. 24, 2008 in the USPTO, the disclosure of which is incorporated herein by reference.

### BACKGROUND

The present invention relates to a novel mutation in the SCN5A gene associated with loss of cardiac sodium channel function.

### RELATED ART

Brugada syndrome (BS) is an inherited electrical cardiac disorder characterized by an incomplete right bundle branch block (RBBB), a typical electrocardiogram (ECG) pattern of ST-segment elevation in the right precordial leads V₁ to V₃, a structurally normal heart, and a highly increased risk of sudden cardiac death as a result of polymorphic ventricular tachycardia (VT) or ventricular fibrillation (VF) (Brugada and Brugada, 1992; Antzelevitch et al., 2002). This disorder is not related to acute ischemia, electrolyte abnormalities or structural heart diseases (Wilde et al., 2002). BS is a familial disease with autosomal dominant transmission. While BS has been observed worldwide, it is more common in Southeast Asian and Japanese populations (Nademanee et al., 1997) and, interestingly, manifests much more frequently in men than women (Priori et al., 2002). The mean age at onset of clinical events (syncope or cardiac arrest) is 30-40 years, although severe forms with earlier onset and even neonatal expression have been reported (Priori et al., 2000).

The cardiac sodium channel is responsible for the generation of the rapid upstroke of the cardiac action potential and plays a key role in cardiac impulse propagation (Balser, 1999). Sodium channels are heterodimeric assemblies composed of a pore forming α-subunit and several regulatory β-subunits. The α-subunit consists of four homologous domains (DI-DIV). Each domain contains six transmembrane segments (S1-S6) connected by short linking intracellular segments (Cohen and Barchi, 1992). The SCN5A gene that encodes the α-subunit of the human cardiac voltage-gated sodium channel (Naᵥ1.5) (Antzelevitch et al., 2002) is located on chromosome 3p21 and consists of 28 exons spanning approximately 80 kb (Wang et al., 1996). While several candidate genes are considered plausible, thus far BS has been linked only to mutations in SCN5A. Understanding the molecular and cellular mechanisms leading to BS remains limited, and, to date, a minority (approximately 20%) of patients with BS have been found to carry a mutation in this gene (Priori et al., 2000). Several reports have estimated the prevalence of Brugada-type ECG changes at approximately 0.1-0.7% in the general population worldwide (Hermida et al., 2000; Matsuo et al., 2001). Genetic studies have demonstrated that some cases of BS and chromosome 3-linked long-QT syndrome (LQT3) are allelic variations of SCN5A.

To date, several dozen SCN5A mutations in patients with BS have been identified. Three categories of SCN5A mutations have been reported in BS: missense, splice-donor, and frameshift (Chen et al., 1998; Deschênes et al., 2000; Naccarelli et al., 2001). Functional analyses have revealed that most SCN5A mutations lead to a loss of function of cardiac sodium channels by reducing the sodium current (I_{Na}) available during the early phase of the cardiac action potential (Balser, 1999; Baroudi et al., 2000). Because I_{Na} plays an important role in human heart excitation and contraction, functional variations in these sodium channels can cause variable cardiac biophysical abnormalities (Wang et al., 2000). Studies conducted over the past decade have shown that rebalancing the currents active at the end of phase 1, which leads to an accentuation of the action potential notch in the right ventricular epicardium, is responsible for the accentuated J-wave or ST segment elevation linked with BS (Antzelevitch, 2001). Yan and Antzelevitch (1999) have suggested that a decrease in the depolarizing inward sodium current leads to early repolarization in the right ventricular epicardium where the transient outward K⁺ current (Iₜₒ) is large. This causes a voltage gradient from endocardium to epicardium, ST elevation on the ECG, and susceptibility to arrhythmias caused by phase 2 re-entry.

Throughout this application, various patents and publications are referenced and citations are provided in parentheses. The disclosures of these patents and publications are hereby incorporated by reference into this application in their entities in order to more fully describe this invention and the state of the art to which this invention pertains.

### SUMMARY

The present inventors have performed intensive research to reveal the genetic background underlying cardiac diseases or disorders caused by loss of cardiac sodium channel function, particularly Brugada syndrome. As a result, we have discovered that a novel heterozygous nonsense mutation in exon 20 of the SCN5A gene is closely related to cardiac diseases or disorders caused by loss of cardiac sodium channel function.

Accordingly, it is an object of this invention to provide an isolated nucleic acid molecule encoding a mutant SCN5A protein.

It is another object of this invention to provide a mutant SCN5A protein.

It is still another object of this invention to provide a method for detecting a cardiac disease or disorder associated with loss of cardiac sodium channel function.

Other objects and advantages of the present invention will become apparent from the following detailed description together with the appended claims and drawings.

### BRIEF DESCRIPTION OF THE DRAWINGS

FIG. 1 Pedigree structure of a family (KBSF3) with BS. Circles indicate females; squares indicate males. Filled symbols indicate affected individuals; symbols with a central point indicate asymptomatic W1191X mutation carriers. The proband with BS is marked by an arrow.

FIGS. 2A-2C Telemetry monitoring on the second day of hospitalization. (FIG. 2A) Polymorphic ventricular tachycardia (PMVT) was documented, and the proband had a syncope. PMVT was terminated spontaneously. (FIG. 2B) Magnified view of box in (FIG. 2A). Polymorphic ventricular tachycardia was induced by PVC without previous a pause. (FIG. 2C) Telemetry monitoring of the proband's daughter (III-2) during a flecainide challenge test. A four-second sinus pause (upper arrow) and RBBB pattern (lower arrow) were observed after the infusion of 20 mg of flecainide.

FIGS. 3A-3B ECG recordings of the two patients in a Korean family (KBSF3) with BS with different patterns in precordial leads V₁ to V₃. (FIG. 3A) ECG patterns of the proband (II-2). Dynamic ECG changes without a drug challenge test for one month are shown. (FIG. 3B) ECG recordings of the proband's daughter (III-2). The ECG pattern in the left panel shows the baseline condition, which converts to the coved type and severe sinus dysfunction after a flecainide challenge test as shown in the right panel. The arrows in the two panels indicate the J-wave.

FIGS. 4A-4C Genetic analysis of the two individuals with SCN5A mutations. (FIG. 4A) Direct DNA sequencing analysis of SCN5A exon 20 from a proband (II-2). The DNA sequence indicates a non-sense mutation (G-to-A base substitution), which results in a tryptophan-to-stop codon substitution at amino acid position 1191. (FIG. 4B) DNA sequence electropherogram shows a missense mutation (S1710L) in SCN5A exon 28 in a patient with BS (SV525). (FIG. 4C) DHPLC confirms an abnormal elution profile in affected cases (II-2 and III-2), two asymptomatic carriers (IV-1 and IV-2) in a family, and 200 unrelated control subjects.

FIG. 5 Schematic membrane topology of the human cardiac sodium channel. The locations of the W1191X and S1710L mutations are indicated by circles and arrows.

FIGS. 6A-6C Representative traces of a family of whole-cell sodium currents from (FIG. 6A) Naᵥ1.5/WT, (FIG. 6B) Naᵥ1.5/W1191X expressed, and (FIG. 6C) Naᵥ1.5/WT+Naᵥ1.5/W1191X coexpressed in the tsA201 cell line. Currents were generated from a holding potential of −140 mV from −80 to +50 mV for 30 ms in 10 mV increments.

FIGS. 7A-7C (FIG. 7A) Current-voltage (I/V) relationships of Naᵥ1.5/WT, Naᵥ1.5/W1191X and Naᵥ1.5/WT+Naᵥ1.5/W1191X, where peak current amplitude was plotted against voltage. (FIG. 7B) Current densities of Naᵥ1.5/WT, Naᵥ1.5/W1191X and Naᵥ1.5/WT+Naᵥ1.5/W1191X. A 50% reduction in sodium channel density for Naᵥ1.5/WT+Naᵥ1.5/W1191X and no expression for Naᵥ1.5/W1191X. (FIG. 7C) Steady-state activation and inactivation of Naᵥ1.5/WT (Δ for activation, n=7; ◯ for inactivation, n=7) and Naᵥ1.5/WT co-expressed with Naᵥ1.5/W1191X (Δ for activation, n=6; ◯ for inactivation, n=6). For activation, V_{1/2}=−55.52±2.48 mV and kᵥ=−6.75±0.35 mV for Naᵥ1.5/WT, and V_{1/2}=−56.51±4.24 mV and kᵥ=−5.97±0.35 mV for Naᵥ1.5/WT co-expressed with Naᵥ1.5/W1191X. For inactivation, V_{1/2}=−105.82±1.58 and kᵥ=4.65±0.22 for Naᵥ1.5/WT, and V_{1/2}=−104.18±2.09 and kᵥ=4.73±0.06 for Naᵥ1.5/WT co-expressed with Naᵥ1.5/W1191X. Activated currents were generated from a holding potential of −140 mV following 50 ms voltage steps from −100 mV to +20 mV in 10 mV increments. The voltage-dependence of inactivation was obtained by measuring the peak Na⁺ current during a 20 ms test pulse to −30 my, which followed a 500 ms pre-pulse to membrane potentials between −140 and −30 mV from a holding potential of −140 my.

### DETAILED DESCRIPTION

In one aspect of this invention, there is provided an isolated nucleic acid molecule encoding a mutant SCN5A protein corresponding to the wild type human SCN5A protein set forth in SEQ ID NO:2, wherein the mutant SCN5A protein has a W1191X mutation.

The present inventors have performed intensive research to reveal the genetic background underlying cardiac diseases or disorders caused by loss of cardiac sodium channel function, particularly Brugada syndrome. As a result, we have discovered that a novel heterozygous nonsense mutation in exon 20 of the SCN5A gene is closely related to cardiac diseases or disorders caused by loss of cardiac sodium channel function.

The term used herein “nucleic acid molecule” includes genomic DNA (gDNA), cDNA and mRNA.

According to a preferred embodiment, the nucleic acid has the sequence corresponding to that of the wild type human SCN5A cDNA set forth in SEQ ID NO:1 with a G to A substitution at nucleotide 3573. The G to A substitution at nucleotide 3573 causes the generation of a stop codon “tga” that is responsible for a change from a tryptophan (W) to a stop codon at position 1191 in the protein.

In another aspect of this invention, there is provided a mutant SCN5A protein consisting of the amino acid sequence set forth in SEQ ID NO:2.

The mutation causing the prematurely truncated form of the wild type SCN5A protein removes domains III and IV and leads to loss of cardiac sodium channel function via haploinsufficiency.

Analysis of the novel mutation provides an early diagnosis of subjects with cardiac diseases or disorders caused by loss of cardiac sodium channel function, particularly Brugada syndrome. Diagnostic methods include analyzing the sequences of the SCN5A gene or protein of an individual to be tested and comparing them with the sequences of the native, nonvariant SCN5A gene or protein. Pre-symptomatic diagnosis of these syndromes will enable practitioners to treat these disorders using existing medical therapy, e.g., using sodium channel blockers or through electrical stimulation.

In further aspect of this invention, there is provided a method for detecting a cardiac disease or disorder associated with loss of cardiac sodium channel function in a subject, which comprises the steps of obtaining a biological sample from the subject, and detecting in the biological sample the presence or the expression of the nucleic acid molecule encoding the mutant SCN5A protein, wherein the detection of the presence or expression of the nucleic acid molecule encoding the mutant SCN5A protein is indicative of the cardiac disease or disorder associated with loss of cardiac sodium channel function.

The biological sample used in the present invention includes any biological sample such as tissue, cell, whole blood, serum, plasma, peripheral blood leukocyte, saliva, semen, urine, synovia and spinal fluid and may be pretreated for assay.

The present method may be carried out at protein, DNA or mRNA level. Where it is performed to detect the mutant SCN5A protein, antibodies to specifically recognize the mutant SCN5A protein are used and the detection is carried out by contacting the biological sample to the antibody specific to the mutant SCN5A protein and evaluating a formation of antigen-antibody complex. The evaluation on antigen-antibody complex formation may be carried out using immunohistochemical staining, radioimmuno assay (RIA), enzyme-linked immunosorbent assay (ELISA), Western blotting, immunoprecipitation assay, immunodiffusion assay, complement fixation assay, FACS and protein chip assay. The evaluation on antigen-antibody complex formation may be performed qualitatively or quantitatively, in particular, by measuring signal from detection label.

The label to generate measurable signal for antigen-antibody complex formation includes, but not limited to, enzyme, fluorophore, ligand, luminophore, microparticle, redox molecules and radioisotopes. The enzymatic label includes, but not limited to, β-glucuronidase, β-D-glucosidase, β-D-galactosidase, urease, peroxidase, alkaline phosphatase, acetylcholinesterase, glucose oxidase, hexokinase, GDPase, RNase, luciferase, phosphofructokinase, phosphoenolpyruvate carboxylase, aspartate aminotransferase, phosphoenolpyruvate decarboxylase, β-lactamase. The fluorescent label includes, but not limited to, fluorescein, isothiocyanate, rhodamine, phycoerythrin, phycocyanin, allophysocyanin, o-phthalate and fluorescamine. The ligand serving as a label includes, but not limited to, biotin derivatives. Non-limiting examples of the luminescent label includes acridinium ester, luciferin and luciferase. Microparticles as label include colloidal gold and colored latex, but not limited to. Redox molecules for labeling include ferrocene, lutenium complex compound, viologen, quinone, Ti ion, Cs ion, diimide, 1,4-benzoquinone, hydroquinone, K₄ W(CN)₈, [Os(bpy)₃]²⁺, [Ru(bpy)₃]²⁺ and [Mo(CN)₈]⁴⁻, but not limited to. The radioisotopes includes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I and ¹⁸⁶Re, but not limited to.

Where the present method is performed to detect the mutant SCN5A mRNA, the detection step may be carried out by an amplification reaction or a hybridization reaction well-known in the art.

The phrase “detection of the mutant SCN5A mRNA” used herein is intended to refer to analyze the existence or amount of the mutant SCN5A mRNA as cardiac disease diagnosis marker in cells by use of primer or probe specifically hybridized with the mutant SCN5A mRNA.

The term “primer” used herein means an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of four different nucleoside triphosphates and a thermostable enzyme in an appropriate buffer and at a suitable temperature.

The term “probe” used herein refers to a linear oligomer of natural or modified monomers or linkages, including deoxyribonucleotides, ribonucleotides and the like, which is capable of specifically hybridizing with a target nucleotide sequence, whether occurring naturally or produced synthetically. The probe used in the present method may be prepared in the form of oligonucleotide probe, single-stranded DNA probe, double-stranded DNA probe and RNA probe. It may be labeled with biotin, FITC, rhodamine, DIG and radioisotopes.

The method to detect the mutant SCN5A mRNA using either primer or probe includes, but not limited to, DNA sequencing, RT-PCR (reverse transcription-polymerase chain reaction), primer extension method (Nikiforov, T. T. et al., Nucl Acids Res 22, 4167-4175 (1994)), oligonucleotide ligation analysis (OLA) (Nickerson, D. A. et al., Pro Nat Acad Sci USA, 87, 8923-8927 (1990)), allele-specific PCR (Rust, S. et al., Nucl Acids Res, 6, 3623-3629 (1993)), RNase mismatch cleavage (Myers R. M. et al., Science, 230, 1242-1246 (1985)), single strand conformation polymorphism (SSCP; Orita M. et al., Pro Nat Acad Sci USA, 86, 2766-2770 (1989)), simultaneous analysis of SSCP and heteroduplex (Lee et al., Mol Cells, 5:668-672 (1995)), denaturation gradient gel electrophoresis (DGGE; Cariello N F. et al., Am J Hum Genet, 42, 726-734 (1988)) and denaturing high performance liquid chromatography (D-HPLC, Underhill P A. et al., Genome Res, 7, 996-1005 (1997)).

Preferably, the method by amplification reaction is carried out by RT-PCR using a primer capable of differentiating an mRNA of the mutant SCN5A from an mRNA of the wild SCN5A. RT-PCR process suggested by P. Seeburg (1986) for RNA research involves PCR amplification of cDNA obtained from mRNA reverse transcription. For amplification, a primer pair specifically annealed to the mutant SCN5A cDNA is used. Preferably, the primer is designed to generate two different sized bands in electrophoresis in which one is specific to the wild SCN5A mRNA and the other to the mutant SCN5A mRNA. Alternatively, the primer is designed to generate only electrophoresis band specific to the mutant SCN5A mRNA.

The amplification reactions using primers may be carried out in accordance with well-known methods. The nucleic acid molecule may be either DNA or RNA. The molecule may be in either a double-stranded or single-stranded form. Where the nucleic acid as starting material is double-stranded, it is preferred to render the two strands into a single-stranded or partially single-stranded form. Methods known to separate strands includes, but not limited to, heating, alkali, formamide, urea and glycoxal treatment, enzymatic methods (e.g., helicase action), and binding proteins. For instance, strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, an oligonucleotide dT primer hybridizable to poly A tail of mRNA is used. The oligonucleotide dT primer is comprised of dTMPs, one or more of which may be replaced with other dNMPs so long as the dT primer can serve as primer. Reverse transcription can be done with reverse transcriptase that has RNase H activity. If one uses an enzyme having RNase H activity, it may be possible to omit a separate RNase H digestion step by carefully choosing the reaction conditions.

The primer used for the present invention is hybridized or annealed to a site on the template such that double-stranded structure is formed. Conditions of nucleic acid annealing suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the amplification step of the present methods, which includes “Klenow” fragment of E coli DNA polymerase I, a thermostable DNA polymerase, and bacteriophage T7 DNA polymerase. Preferably, the polymerase is a thermostable DNA polymerase which may be obtained from a variety of bacterial species, including Thermus aquaticus (Taq), Thermus thermophilus (Tth), Thermus flliformis, Thermis flavus, Thermococcus literalis, and Pyrococcus furiosus (Pfu). Many of these polymerases may be isolated from bacterium itself or obtained commercially. Polymerase to be used with the subject invention can also be obtained from cells which express high levels of the cloned genes encoding the polymerase.

When a polymerization reaction is being conducted, it is preferable to provide the components required for such reaction in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, dATP, dCTP, dGTP, and dTTP in sufficient quantity to support the degree of the extension desired.

All of the enzymes used in this amplification reaction may be active under the same reaction conditions. Indeed, buffers exist in which all enzymes are near their optimal reaction conditions. Therefore, the amplification process of the present invention can be done in a single reaction volume without any change of conditions such as addition of reactants.

Annealing or hybridization in the present method is performed under stringent conditions that allow for specific binding between the primer and the template nucleic acid. Such stringent conditions for annealing will be sequence-dependent and varied depending on environmental parameters.

Most preferably, the amplification is performed in accordance with PCR which is disclosed in U.S. Pat. Nos. 4,683,195, 4,683,202, and 4,800,159.

The analysis of amplified products in the present invention may be conducted by various methods or protocols, e.g. electrophoresis such as agarose gel electrophoresis.

Alternatively, the present method may be carried out in accordance with hybridization reaction using suitable probes.

The stringent conditions of nucleic acid hybridization suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985). As used herein the term “stringent condition” refers to the conditions of temperature, ionic strength (buffer concentration), and the presence of other compounds such as organic solvents, under which hybridization or annealing is conducted. As understood by those of skill in the art, the stringent conditions are sequence dependent and are different under different environmental parameters. Longer sequences hybridize or anneal specifically at higher temperatures.

Some modifications in the probes used in this invention can be made unless the modifications abolish the advantages of the oligonucleotides. Such modifications, i.e., labels linking to the probes generate a signal to detect hybridization. Suitable labels include fluorophores, chromophores, chemiluminescers, magnetic particles, radioisotopes, mass labels, electron dense particles, enzymes, cofactors, substrates for enzymes and haptens having specific binding partners, e.g., an antibody, streptavidin, biotin, digoxigenin and chelating group, but not limited to. The labels generate signal detectable by fluorescence, radioactivity, measurement of color development, mass measurement, X-ray diffraction or absorption, magnetic force, enzymatic activity, mass analysis, binding affinity, high frequency hybridization or nanocrystal.

Preferably, the probes used in the present invention may be immobilized on a solid substrate (nitrocellulose membrane, nylon filter, glass plate, silicon wafer and fluorocarbon support) to fabricate microarray. In microarray, the probes serve as hybridizable array elements.

The probes used in the hybridization reaction have the mutant SCN5A specific nucleotide sequence which is not found in the wile type SCN5A.

The present method may be carried out by direct sequencing of gDNA or mRNA. The general processes for sequencing of nucleic acid molecules are found in Sambrook, J. et al., Molecular Cloning. A Laboratory Manual, 3rd ed. Cold Spring Harbor Press (2001), the teachings of which are incorporated herein by reference in their entity.

The present method is very useful in diagnosing a variety of cardiac diseases and disorders. Preferably, the present method is applied to the detection of Brugada syndrome, long QT syndrome, atrial arrhythmia or progressive conduction disease, more preferably, Brugada syndrome.

Pre-symptomatic diagnosis of these syndromes will enable practitioners to treat these disorders using existing medical therapy, e.g., using sodium channel blockers or through electrical stimulation.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Materials and Methods

Subjects and Clinical Data

Our study population consisted of 34 individuals including 33 members arising from four unrelated families and one patient. Of these, five individuals, one from each family and the one patient, were initially diagnosed with BS. All were recruited from the Yonsei Cardiovascular Genome Center (Republic of Korea). The protocol for this study was approved by the Ethics Committee of Yonsei University and the study was carried out under its guidelines. Informed consent was obtained from all participants.

The diagnosis of BS was based on a 12-lead electrocardiogram (ECG) with the following ECG parameters: (1) at least a 2 mm ST-segment elevation in more than one right precordial lead (V₁ to V₃) with an RBBB morphology, (2) a J-wave elevation >0.2 mV at baseline, and (3) the presence of a structural abnormality of the heart as evaluated by echocardiography. Three types of ST-segment elevation patterns are recognized. Type 1 is characterized by a prominent coved ST-segment elevation displaying J wave amplitude or a ST-segment elevation ≧2 mm or 0.2 mV at its peak followed by a negative T-wave, with little or no isoelectric separation. Type 2 also has a high take-off ST-segment elevation, but in this case, J-wave amplitude (≧2 mm) gives rise to a gradually descending ST-segment elevation (remaining ≧1 mm above the baseline), followed by a positive or biphasic T-wave that results in a saddleback configuration. Type 3 is characterized by a right precordial ST-segment saddleback and/or coved elevation of <1 mm (Wilde et al., 2002). A challenge test was performed in a subset of family members by infusing 2 mg/kg of flecainide for 10 min. Two hundred individuals with no a history of structural heart disorder, heart failure, syncope, VF, or ventricular tachycardia (VT) were recruited as unrelated healthy control subjects.

Genetic Analysis of SCN5A

The mutation analysis was carried out by polymerase chain reaction (PCR) followed by direct sequencing. Genomic DNAs from the probands as well as a normal subject from each family were sequenced for the all coding regions and the flanking introns of SCN5A. Genomic DNA was extracted from peripheral blood leukocytes using QIAamp® DNA blood kits (Qiagen, Valencia, Calif.). The PCR was performed using modified primers located in the intronic sequences and amplification conditions as previously described (Wang et al., 1996).

The amplified products were purified using QIAquick® PCR purification kits (Qiagen) and directly sequenced using ABI PRISM Big Dye Terminator Cycle Sequencing Ready Reaction kits and an ABI PRISM 3100 DNA analyzer (Applied Biosystems, Foster City, Calif.). Sequences were compared with the reference genomic and cDNA sequences of SCN5A (GenBank accession numbers NT 022517.17 and AY148488.1, respectively) using BLASTN. Once the mutation was identified, the PCR product was used to determine denaturing high-performance liquid chromatography (DHPLC) conditions. A DHPLC analysis with a WAVE™ System Model 3500 (Transgenomic, Omaha, Nebr.) was used to detect for sequence variations in a control group of 200 individuals from the same ethnicity (Korean) as previously reported (Underhill et al., 1997; Ackerman et al., 2001). DHPLC was performed on DNA amplification products using optimal temperature conditions (at 60° C.). Sequences underlying abnormal DHPLC profiles were validated by reamplification of the same genomic DNA and were analyzed by direct DNA sequencing as described above.

Site-Directed Mutagenesis

Mutant Naᵥ1.5/W1191X was generated using QuickChange™ site-directed mutagenesis kits according to the manufacturer's instructions (Stratagene, La Jolla, Calif.). The Naᵥ1.5/mutants were constructed using the following 33-nucleotide mutagenic sense and antisense primers:

5′-GCC CCA GGG AAG GTC TGA TGG CGG TTG CGC AAG-3′

5′-CTT GCG CAA CCG CCA TCA GAC CTT CCC TGG GGC-3′

Mutated sites are underlined. Mutant and WT Naᵥ1.5 in a pcDNA1 construct were purified using Qiagen columns (Qiagen).

Transfection of the tsA201 Cell Line

The tsA201 cells (human embryonic kidney cell line, Chang C C et al., A novel SCN5A mutation manifests as a malignant form of long QT syndrome with perinatal onset of tachycardia/bradycardia. (2004). Cardiovascular Research 64(2): 268-278) were grown in high glucose DMEM supplemented with FBS (10%), L-glutamine (2 mM), penicillin (100 U/ml) and streptomycin (10 mg/ml) (Gibco BRL Life Technologies, Burlington, ON, Canada) and were incubated in a 5% CO₂ humidified atmosphere. The cells were transfected using the calcium phosphate method (Margolskee et al., 1993) with the following modification to facilitate the identification of individual transfected cells: cells were cotransfected with the expression vector piERS/CD8/β₁ which conferred expression of the β₁-subunit as well as a lymphocyte surface antigen (CD8-a) (Jurman et al., 1994). Using this strategy, we were able to select for transfected cells using anti-CD8-a coated beads. Five micrograms of plasmid DNA coding for WT or mutant Na⁺ channels, and 5 μg of piERS/CD8/β₁ were used. For patch clamp experiments, 2 to 3-day-post-transfection cells were incubated for 5 min in a medium containing anti-CD8-a coated beads (Dynabeads M-450 CD8-a) (Jurman et al., 1994). Unattached beads were removed by washing. The beads were prepared according to the manufacturer's instructions (Dynal, Oslo, Norway). Cells expressing CD8-a on their surface fixed the beads and were visually distinguishable from nontransfected cells by light microscopy.

Patch-Damp Method

Macroscopic Na⁺ currents from tsA201-transfected cells were recorded using the whole-cell configuration of the patch-clamp technique (Hamill et al., 1981). Patch electrodes were made from 8161 Corning borosilicate glass and coated with Sylgard (Dow-Corning, Midland, Mich.) to minimize their capacitance. Patch clamp recordings were made using low resistance electrodes (<1 MΩ), and a routine series resistance compensation by an Axopatch 200 amplifier (Axon Instruments, Foster City, Calif.) was performed to values >80% to minimize voltage-clamp errors. Voltage-clamp command pulses were generated by microcomputer using pCLAMP software v8.0 (Axon Instruments). Na⁺ currents were filtered at 5 kHz, digitized at 10 kHz, and stored on a microcomputer equipped with an AD converter (Digidata 1300, Axon Instruments). Data analysis was performed using a combination of pCLAMP software v9.0 (Axon Instruments), Microsoft Excel and SigmaPlot 2001 for Windows v7.0 (SPSS Chicago, Ill.).

Solutions and Reagents

For whole cell recordings, the patch pipette contained 35 mM NaCl, 105 mM CsF, 10 mM EGTA, and 10 mM Cs-HEPES. The pH was adjusted to 7.4 using 1 N CsOH. The bath solution contained 150 mM NaCl, 2 mM KCl, 1.5 mM CaCl₂, 1 mM MgCl₂, 10 mM glucose, and 10 mM Na-HEPES. The pH was adjusted to 7.4 with 1 N NaOH. A −7 mV correction of the liquid junction potential between the patch pipette and the bath solutions was performed. The recordings were made 10 min after obtaining the whole cell configuration in order to allow the current to stabilize and achieve adequate diffusion of the contents of the patch electrode. Experiments were carried out at room temperature (22-23° C.).

Results

Clinical Characteristics

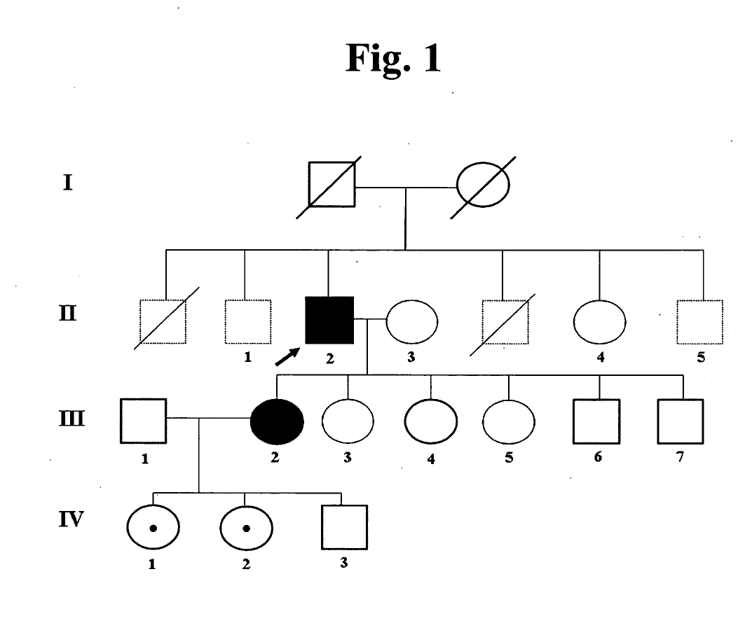

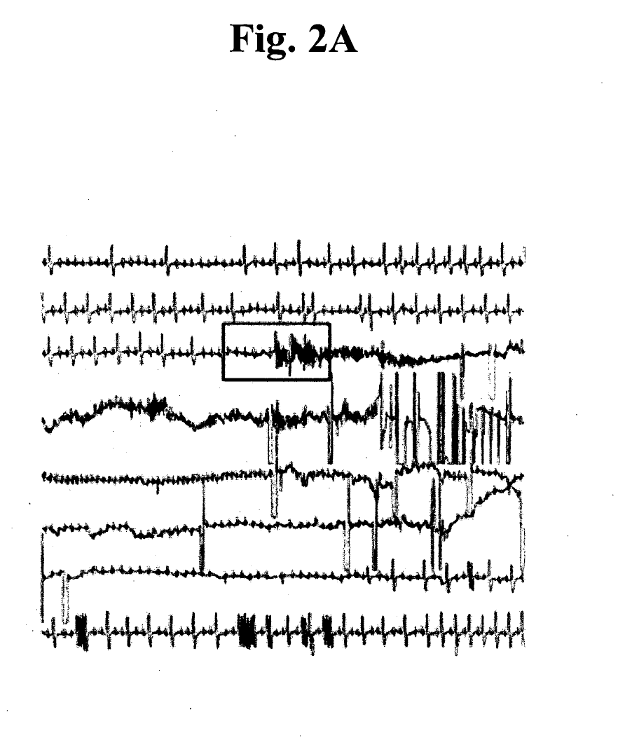

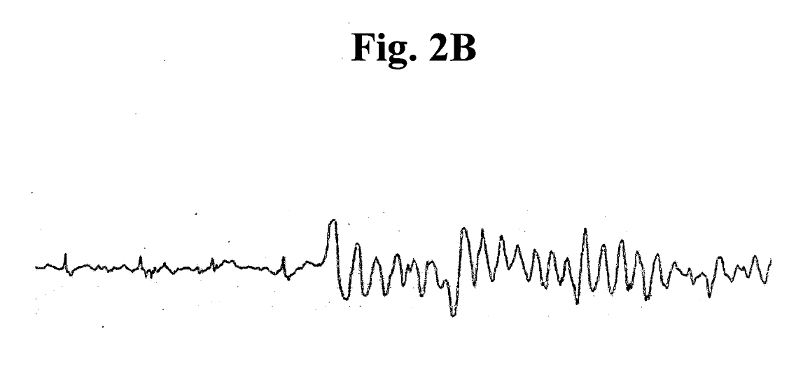

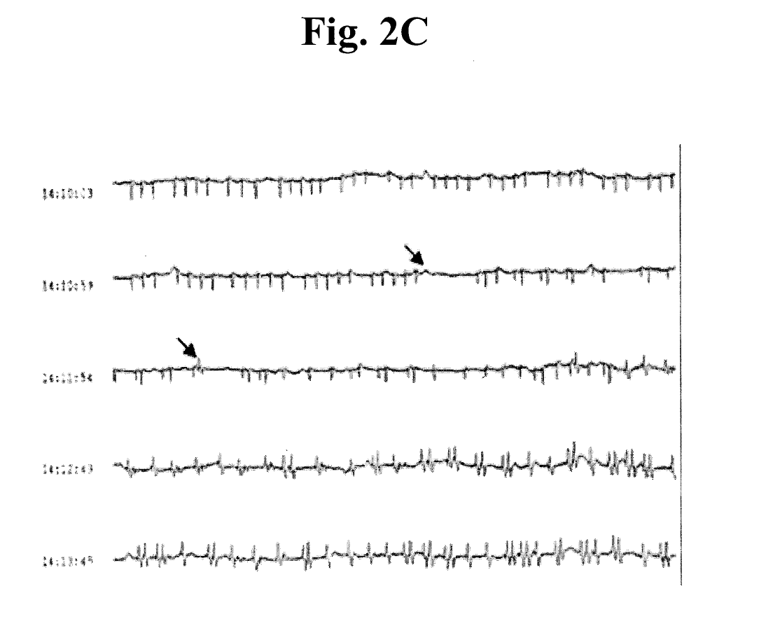

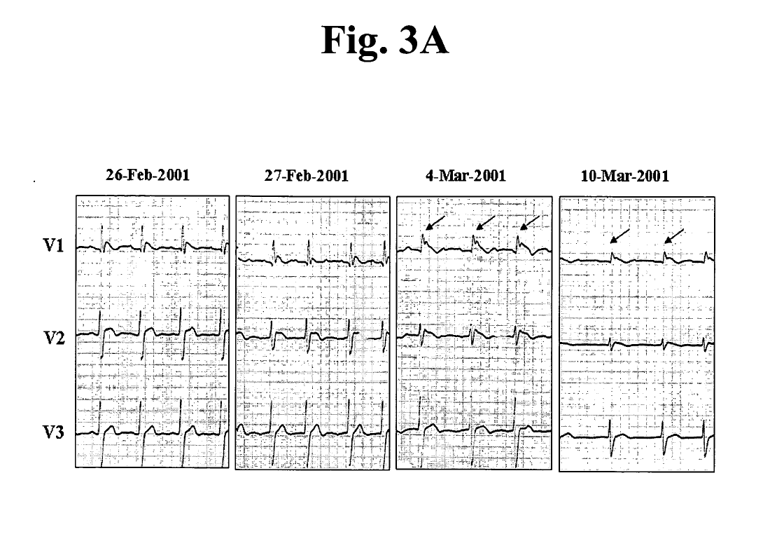

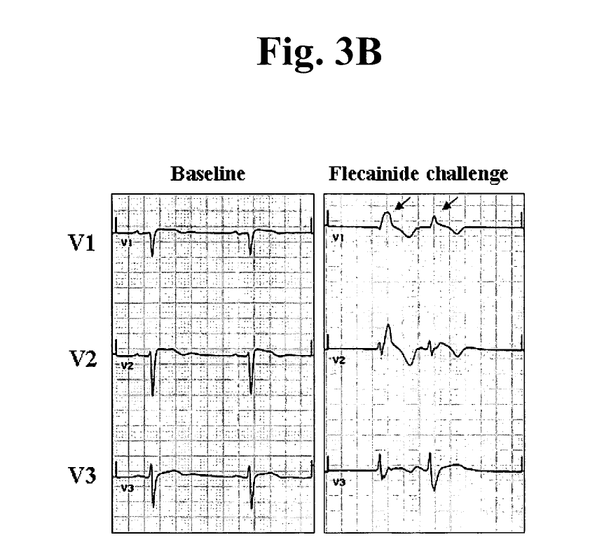

Four probands and one patient with a type 1 BS-type ECG (all males; 22-74 years of age) were enrolled in our study. One proband, a 74-year-old man (II-2, FIG. 1) from the KBSF3 family, had been initially referred with two episodes of syncope that had occurred on Feb. 23 and 26, 2001. An ECG of atrial flutter 1:1 conduction with aberrant conduction was documented on Feb. 27, 2001. He had undergone bidirectional isthmic conduction block in May 2001. At that time, VF was induced by a triple ventricular extrastimulation of 500/270/230/240 ms at apex. Because VF can be induced by triple extrastimulation with an idiosyncratic response, further management for VF was not performed. However, he was hospitalized with a recurrent syncope on Dec. 18, 2003. He had a syncope again on the second day of hospitalization, and polymorphic ventricular tachycardia (PMVT) was documented by telemetry monitoring (FIG. 2A-B). The ECG recordings from the proband showed the dynamic ECG changes (FIG. 3A). He had an abnormal prolonged QT interval (467 ms), PR interval (216 ms), and QRS duration (140 ms). Coved ST-segment elevation (type 1) was evident in leads V₁ and V_{1-2—}. He had 14 episodes of VF, which were successfully terminated with shock delivery by ICD. His familial history revealed that his daughter I-2) had had a syncope and several episodes of dizziness with sinus dysfunction. She had showed RBBB and coved-type ST-segment elevation with a 40 mg dose of flecainide as well as severe sinus dysfunction (FIG. 3B). With the exception of III-2, all five offspring of the proband turned out to be clinically normal.

Individual case SV525 had a family history of sudden cardiac death. The initial 12-lead baseline ECG had an asymptomatic pattern but flecainide unmasked a type 1 ECG phenotype (data not shown). An implantable cardioverter defibrillator (ICD) was implanted to prevent sudden cardiac death in all patients with BS.

Genetic Analysis

All the probands and a normal family member from each family were screened for all the exons and flanking introns of SCN5A by PCR-direct DNA sequencing. Once the mutation had been identified in a family, other family members were screened for the mutation by PCR-DHPLC-direct sequencing. PCR-DHPLC was used to determine the absence of the mutation in control subjects. SCN5A mutations were identified in one family (KBSF3) and one patient (SV525), while four polymorphisms were found in the study subjects.

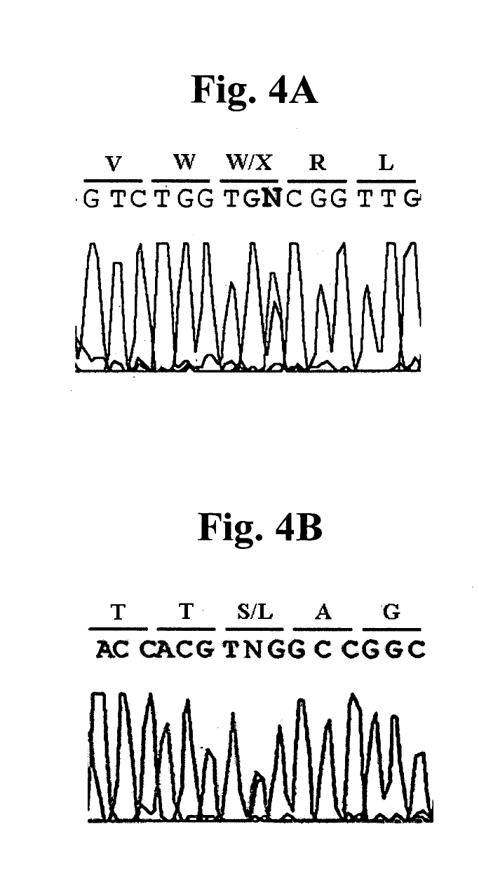

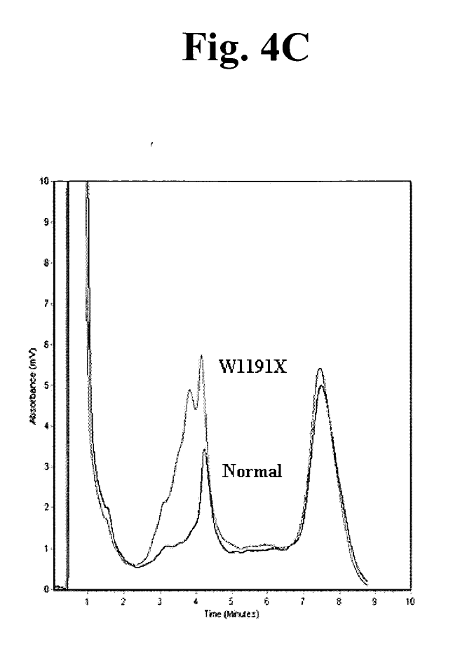

A novel heterozygous nonsense mutation was identified in one family (KBSF3). DNA sequencing analysis of SCN5A in the proband revealed a G-to-A base substitution at position 3973 in exon 20 (FIG. 4A), which presumably causes a change from a tryptophan (W) to a stop codon at position 1191 in the protein (W1191X). Among the family members tested for the mutation (II-2, III-1, III-2, III-4, III-6, III-7, IV-1, IV-2, and IV-3), we confirmed by DHPLC the abnormal conformer in four (II-2, III-2, IV-1, and IV-2), including the proband (FIG. 4C). Sequencing analysis confirmed that W1191X was heterozygous that these individuals had no other SCN5A mutations. Two granddaughters of the proband (IV-1 and IV-2) had the W1191X mutation. However, their ECGs exhibited normal patterns after administration of flecainide.

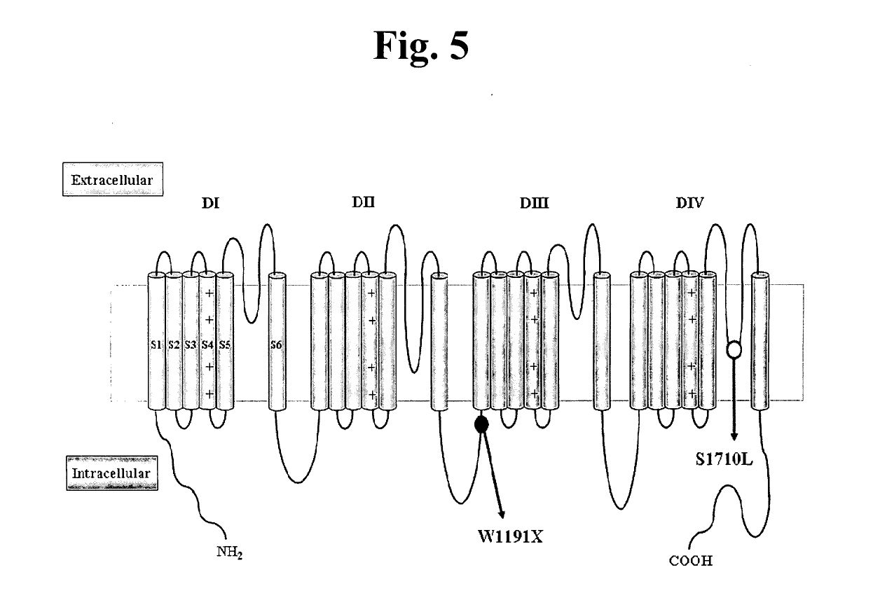

We also identified a previously reported BS SCN5A mutation (S1710L) (FIG. 4B) in a patient with BS (SV525) (Akai et al., 2000). While this mutation was found in an idiopathic ventricular fibrillation (IVF) patient who did not have a typical Brugada ECG pattern, the heterologously expressed S1710L mutant sodium channel is known to present severe alterations in channel inactivation and activation that result in decreased sodium current amplitude (Akai et al., 2000). This mutation is located in the S5-S6 linker that forms part of the channel pore (FIG. 5). The W1191X and S1710L mutations were not found in unrelated normal control individuals or any other study subjects. In addition, four single nucleotide polymorphisms (SNPs) [A29A (G87A; exon 2), H558R (A1673G; exon 12), D1818D (T5454C; exon 28) and IVS24 +53T>C (intron 24)] were found in both control subjects and BS patients. These variants have been previously reported in several ethnic populations (Iwasa et al., 2000; Viswanathan et al., 2003; Chen et al., 2004; Maekawa et al., 2005). The allelic frequencies of these SNPs (29A, 558R, 1818D and IVS24 +53C) in 150 normal Korean controls were 0.263, 0.167, 0.333, and 0.687, respectively.

Biophysical Analysis of SCN5A W1191X

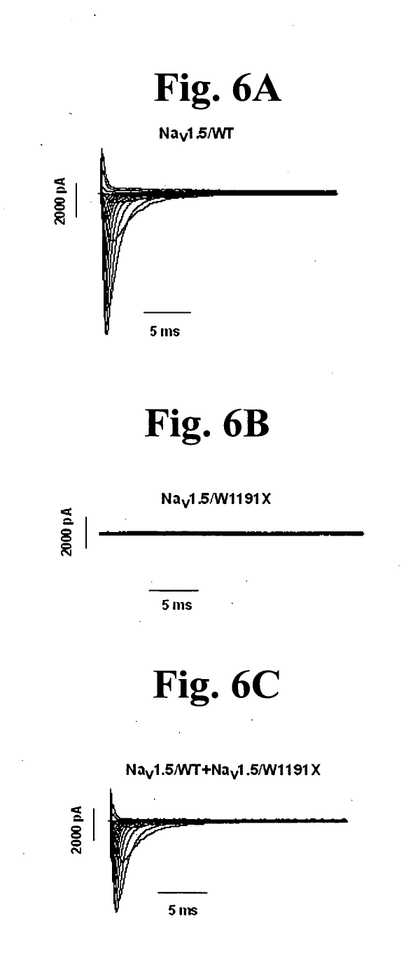

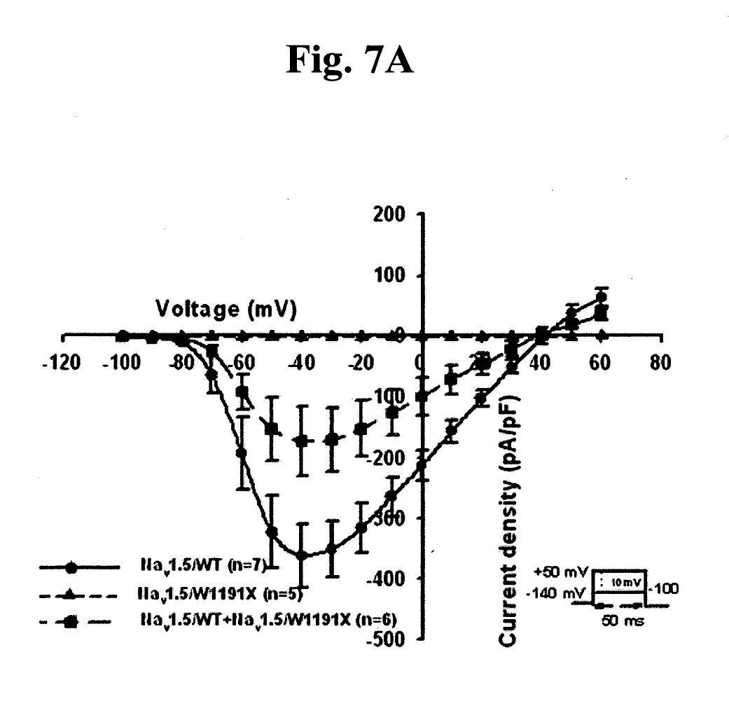

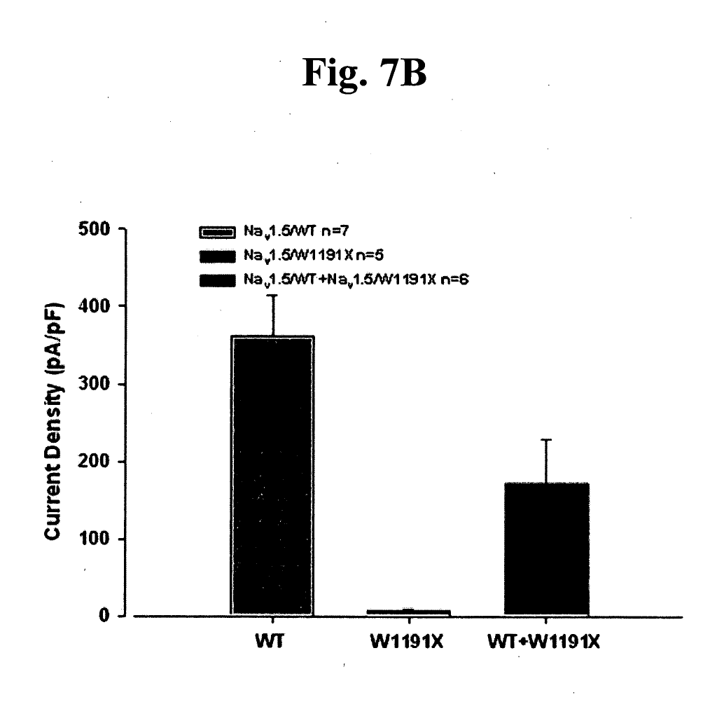

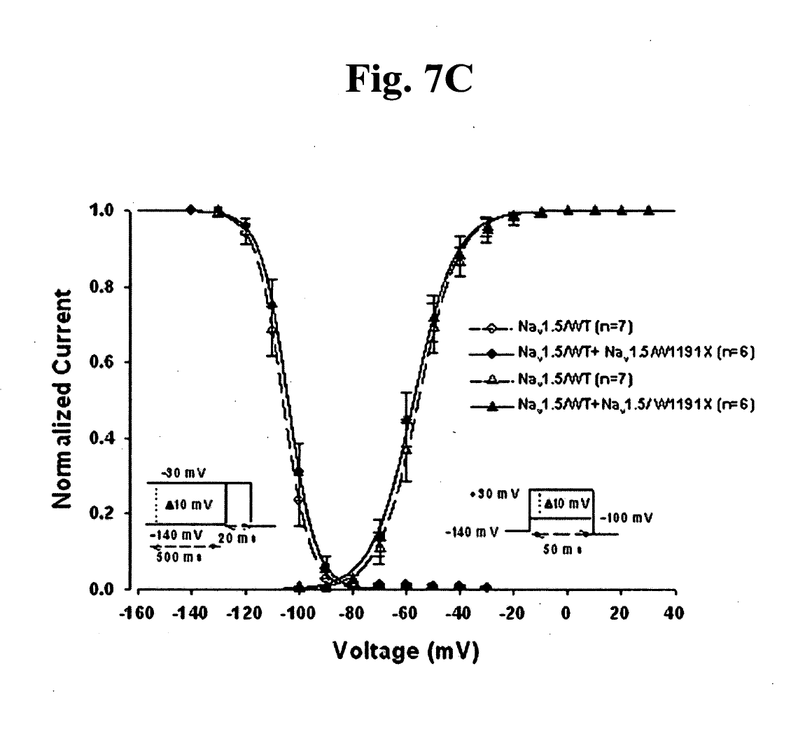

Macroscopic sodium currents were recorded from tsA201 cells expressing either WT (Naᵥ1.5/WT) or mutant channels (Naᵥ1.5/W1191X) co-transfected with the β₁-subunit (see Materials and Methods for more details on identifying cells expressing the β₁-subunit) (FIG. 6). Mutant Naᵥ1.5/W1191X, did not exhibit any currents (FIGS. 6B and 7A). The resulting sodium currents from WT, and WT co-expressed with W1191X, had fast activation and inactivation kinetics (FIG. 6C). Furthermore, co-expression of the WT channel and the mutant channel resulted in a 50% reduction in sodium currents (FIG. 7B), suggesting that there is no dominant negative effect of the mutation. No significant effect on steady-state activation and inactivation was observed (FIG. 7C).

Discussion

In this report, we investigated the genetic and biophysical characteristics of the SCN5A gene in Korean BS patients and control subjects. The BS patients had typical ECGs with RBBB and ST-segment elevation in leads V₁ through V₃ both before and after the administration of a sodium channel blocker. They had no structural heart abnormalities. The clinical and ECG criteria were based on those previously reported (Brugada and Brugada, 1997; Wilde et al., 2002).

To date, a number of SCN5A mutations associated with BS have been reported (Chen et al., 1998; Deschênes et al., 2000; Makiyama et al., 2005). We identified two mutations in the SCN5A gene in Korean BS patients. One of these mutations was a novel heterozygous nonsense mutation (W1191X), which to our knowledge, has not been previously reported in any ethnic group. This mutation occurred in the linker between domains II and III of SCN5A, a few residues upstream from the boundary of the S1 transmembrane segment in domain III. This residue is highly conserved in various mammalian sodium channel isoforms. The functional significance of the linker is not yet clear. However, a recent study of a mutation in this linker region has shown that the voltage dependence of steady state activation remains unchanged while inactivation displays a negative shift (Wang et al., 2004). The W1191X mutation was inherited as an autosomal dominant trait in this family. The same SCN5A mutation was found in four individuals in the family of the proband. Other mutations of this gene were not detected in this family. In addition, the mutation was absent in 200 unrelated normal subjects. The proband (II-2), a 74-year-old man, and his 52-year-old daughter (III-2) had typical Brugada-type ECG patterns and carried the same heterozygous mutant allele. Of interest is the fact that this proband had atrial arrhythmia (AA), a prolonged QTc interval, and first degree A-V block. QTc prolongation and first degree A-V block in the index patients were not related to cardioversion, radiofrequency ablation of atrial flutter, myocardial ischemia or medication. This suggests that SCN5A mutations may lead to both type 3 LQT syndrome and BS. In addition, since his daughter also had AA, the AA in the proband might be related to the SCN5A mutation. However, we could not confirm that the AA in this BS patient was related to a genetic problem, since it is not uncommon in patient his age.

Two granddaughters of the proband, a 27-year-old woman (IV-1) and her 25-year-old sister (IV-2), both had the W1191X mutation. However, their ECGs did not show Brugada-type patterns, and flecainide challenges did not unmask the type 1 BS ECG pattern. Priori et al. (2000) reported false negative results when flecainide and procainamide are used to unmask the syndrome.

One possible explanation for the negative response in the presence of an SCN5A mutation is the incomplete penetrance of BS that appears to be dependent on age and sex. Indeed, Schulze-Bahr et al. (2003) reported complete penetrance in adult patients but incomplete penetrance in young subjects. In addition, there is a greater correlation between the phenotypic expression of BS and sex than for other autosomally dominant transmitted arrhythmic diseases. Although mutant alleles responsible for BS are transmitted equally to both sexes, the clinical phenotype is more predominant in males than in females (Priori et al., 2002; Wilde et al., 2002). The basis for this discrepancy between the sexes is unclear. However, a recent study showed a more prominent Iₜₒ-mediated action potential notch in the right ventricular (RV) epicardium of males than of females (Di Diego et al., 2002). This could explain why BS is eight to ten times more prevalent in men than in women. A similar penetrance mode was observed in our family. As such, the concept of age- and sex-dependent ECG findings in BS might be applicable to this family.

Another possible explanation for the variable Brugada-type ECG patterns in this family is that unidentified factors may modulate the BS phenotype expressed by an SCN5A mutation. To test this possibility, we looked for the H558R polymorphism that is known to modulate the biophysical effects of SCN5A mutations (T512I and M1166L) on sodium channel function (Viswanathan et al., 2003; Ye et al., 2003). We found that all the family members including unaffected individuals, were H558 homozygotes, indicating that H558R polymorphism could not be a factor influencing the BS phenotype.

Naᵥ1.5/W1191X resulted in a loss of cardiac sodium channel function. This mutation was predicted to prematurely truncate the sodium channel protein, removing domains III and IV and might have led to a loss of channel function via haploinsufficiency. This concept is supported by other surveys reporting that the haploinsufficiency of the Naᵥ1.5 protein is a plausible explanation for the reduced sodium current (Benson et al., 2003; Keller et al., 2005). In our in vitro experiments, the tsA201 cells transfected with Naᵥ1.5/W1191X did not express a sodium current whereas the co-expression of this mutant with WT channels resulted in a 50% reduction in sodium currents. This suggests that Naᵥ1.5/W1191X did not exert a dominant negative effect that could lead to a serious BS phenotype. This finding is consistent with recent reports demonstrating that some truncated sodium channel proteins can cause BS (Baroudi et al., 2004; Keller et al., 2005; Todd et al., 2005).

We identified a known heterozygous missense SCN5A mutation (S1710L) in a patient with BS (SV525). Akai et al. (2000) reported the S1710L mutation in a symptomatic IVF patient who had no history of a typical BS ECG phenotype. This mutant sodium channel features an acceleration in current decay together with a large hyperpolarizing shift of steady-state inactivation and a depolarizing shift of activation. Our result suggests that the S1710L mutation in SCN5A is related to clinical phenotypes of IVF and BS with variable clinical features. The occurrence of the S1710L mutation in both IVF and BS patients indicates that the BS and IVF subgroups are at least genetically overlap and may be allelic disorders that result from defects in the SCN5A gene such as congenital LQT syndrome (LQT3) and hereditary A-V block (Schott et al., 1999).

Two of the five BS patients in our study had SCN5A mutations, while none were identified in the other three BS patients. Several recent studies have described BS patients with no SCN5A mutations (Smits et al., 2002; Takahata et al., 2003; Shin et al., 2004). These results are consistent with our findings, which provide support for the possibility of genetic heterogeneity in BS (Priori et al., 2000).

In summary, we describe a novel heterozygous non-sense mutation (W1191X) of the SCN5A gene in a Korean family with BS. The biophysical data confirmed that the loss of function caused by the Naᵥ1.5/W1191X mutation led to BS in our patients. This is consistent with the findings of other studies indicating that the loss of function of SCN5A is responsible for the clinical features of BS. We will continue our search for genes responsible for BS in subjects in whom no SCN5A mutations have been identified.

This work was supported by a grant from Korean Research Foundation Grant (KRF-2002-075-C00020) for Dr. D. J. Shin, a grant from Ministry of Health & Welfare, Republic of Korea (A000385) for Dr. S. J. K. Yoon.

Having described at least one preferred embodiment of the present invention, it is to be understood that variants and modifications thereof falling within the spirit of the invention may become apparent to those skilled in this art, and the scope of this invention is to be determined by appended claims and their equivalents.

### REFERENCES

Ackerman, M. J., Siu, B. L., Sturner, W. Q., Tester D. J., Valdivia C. R., Makielski, J. C., Towbin, J. A., 2001. Postmortem molecular analysis of SCN5A defects in sudden infant death syndrome. The Journal of the American Medical Association 286, 2264-2269.
Akai, J., Makita, N., Sakurada, H., Shirai, N., Ueda, K., Kitabatake, A., Nakazawa, K., Kimura, A., Hiraoka, M., 2000. A novel SCN5A mutation associated with idiopathic ventricular fibrillation without typical ECG findings of Brugada syndrome. FEBS Letters 479, 29-34.
Antzelevitch, C., 2001. Electrical heterogeneity, cardiac arrhythmias, and sodium channel. Circulation Research 87, 964-965.
Antzelevitch, C., Brugada, P., Brugada, J., Brugada, R., Shimizu, W., Gussak, I., Perez Riera, A. R., 2002. Brugada syndrome: a decade of progress. Circulation Research 91, 1114-1118.
Balser, J. R., 1999. Structure and function of the cardiac sodium channels. Cardiovascular Research 42, 327-338.
Baroudi, G., Carbonneau, E., Pouliot, V., Chahine, M., 2000. SCN5A mutation (T1620M) causing Brugada syndrome exhibits different phenotypes when expressed in Xenopus oocytes and mammalian cells. FEBS Letters 467, 12-16.
Baroudi, G., Napolitano, C., Priori, S. G., Bufalo, A. D., Chahine, M., 2004. Loss of function associated with novel mutations of the SCN5A gene in patients with Brugada syndrome. Canadian Journal of Cardiology 20, 425-430.
Benson, D. W., Wang, D. W., Dyment, M., Knilans, T. K., Fish, F. A., Strieper, M. J., Rhodes, T. H., George, A. L. Jr., 2003. Congenital sick sinus syndrome caused by recessive mutations in the cardiac sodium channel gene (SCN5A). The Journal of Clinical Investigation 112, 1019-1028.
Brugada, P., Brugada, J., 1992. Right bundle branch block, persistent ST segment elevation and sudden cardiac death: a distinct clinical and electrocardiographic syndrome. A multicenter report. Journal of the American College of Cardiology 20, 1391-1396.
Brugada, J., Brugada, P., 1997. Further characterization of the syndrome of right bundle branch block, ST segment elevation, and sudden cardiac death. Journal of Cardiovascular Electrophysiology Electrophysiol 8, 325-331.
Chen, Q., Kirsch, G. E., Zhang, D., Brugada, R., Brugada, J., Brugada, P., Potenza, D., Moya, A., Borggrefe, M., Breithardt, G., Ortiz-Lopez, R., Wang, Z., Antzelevitch, C., O'Brien, R. E., Schulze-Bahr, E., Keating, M. T., Towbin, J. A., Wang, Q., 1998. Genetic basis and molecular mechanism for idiopathic ventricular fibrillation. Nature 392, 293-296.
Chen, J. Z., Xie, X. D., Wang, X. X., Tao, M., Shang, Y. P., Guo, X. G., 2004. Single nucleotide polymorphisms of the SCN5A gene in Han Chinese and their relation with Brugada syndrome. Chinese Medical Journal 117, 652-656.
Cohen, S. A., Barchi, R. L., 1992. Cardiac sodium channel structure and function. Trends in Cardiovascular Medicine 2, 133-140.
Deschênes, I., Baroudi, G., Berthet, M., Barde, I., Chalvidan, T., Denjoy, I., Guicheney, P., Chahine, M., 2000. Electrophysiological characterization of SCN5A mutations causing long QT (E1784K) and Brugada (R1512W and R1432G) syndromes. Cardiovascular Research 46, 55-65.
Di Diego, J. M., Cordeiro, J. M., Goodrow, R. J., Fish, J. M., Zygmunt, A. C., Perez, G. J., Scornik, F. S., Antzelevitch, C., 2002. Ionic and cellular basis for the predominance of the Brugada syndrome phenotype in males. Circulation 106, 2004-2011.
Hamill, O. P., Marty, A., Neher, E., Sakmann, B., Sigworth, F. J., 1981. Improved patch-clamp techniques for high-resolution current recording from cells and cell-free membrane patches. Pflügers Archiv: European Journal of Physiology 391, 85-100.
Hermida, J. S., Lemoine, J. L., Aoun, F. B., Jarry, G., Rey, J. L., Quiret, J. C., 2000. Prevalence of the Brugada syndrome in an apparently healthy population. American Journal of Cardiology 86, 91-94.

Iwasa, H., Itoh, T., Nagai, R., Nakamura, Y., Tanaka, T., 2000. Twenty single nucleotide polymorphisms (SNPs) and their allelic frequencies in four genes that are responsible for familial long QT syndrome in the Japanese population. Journal of Human Genetics 45, 182-183.
Jurman, M. E., Boland, L. M., Liu, Y., Yellen, G., 1994. Visual identification of individual transfected cells for electrophysiology using antibody-coated beads. Biotechniques 17, 876-881.
Keller, D. I., Barrane, F. Z., Gouas, L., Martin, J., Pilote, S., Suarez, V., Osswald, S., Brink, M., Guicheney, P., Schwick, N., Chahine, M., 2005. A novel nonsense mutation in the SCN5A gene leads to Brugada syndrome and a silent gene mutation carrier state. Canadian Journal of Cardiology 21, 925-931.
Keller, D. I., Rougier, J. S., Kucera, J. P., Benammar, N., Fressart, V., Guicheney, P., Madle, A., Fromer, M., Schlapfer, J., Abriel, H., 2005. Brugada syndrome and fever: genetic and molecular characterization of patients carrying SCN5A mutations. Cardiovascular Research 67, 510-519.
Maekawa, K., Saito, Y., Ozawa, S., Adachi-Akahane, S., Kawamoto, M., Komamura, K., Shimizu, W., Ueno, K., Kamakura, S., Kamatani, N., Kitakaze, M., Sawada, J., 2005. Genetic polymorphisms and haplotypes of the human cardiac sodium channel alpha subunit gene (SCN5A) in Japanese and their association with arrhythmia. Annals of Human Genetics 69, 413-428.
Makiyama, T., Akao, M., Tsuji, K., Doi, T., Ohno, S., Takenaka, K., Kobori, A., Ninomiya, T., Yoshida, H., Takano, M., Makita, N., Yanagisawa, F., Higashi, Y., Takeyama, Y., Kita, T., Horie, M., 2005. High risk for bradyarrhythmic complications in patients with Brugada syndrome caused by SCN5A gene mutations. Journal of the American College of Cardiology 46, 2100-2106.
Margolskee, R. F., McHendry-Rinde, B., Horn, R., 1993. Panning transfected cells for electrophysiological studies. Biotechniques 15, 906-911.
Matsuo, K., Akahoshi, M., Nakashima E., Suyama, A., Seto, S., Hayano, M., Yano, K., 2001. The prevalence, incidence and prognostic value of the Brugada-type electrocardiogram: a population-based study of four decades. Journal of the American College of Cardiology 38, 765-770.
Naccarelli, G. V., Antzelevitch, C., 2001. The Brugada syndrome: clinical, genetic, cellular, and molecular abnormalities. The American Journal of Medicine 110, 573-581. Nademanee, K., Veerakul, G., Nimmannit, S., Chaowakul, V., Bhuripanyo, K., Likittanasombat, K., Tunsanga, K., Kuasirikul, S., Malasit, P., Tansupasawadikul, S., Tatsanavivat, P, 1997. Arrhythmogenic marker for the sudden unexplained death syndrome in Thai men. Circulation 96, 2595-2600.
Priori, S. G., Napolitano, C., Giordano, U., Collisani, G., Memmi, M., 2000. Brugada syndrome and sudden cardiac death in children. Lancet 355, 808-809.
Priori, S. G., Napolitano, C., Gasparini, M., Pappone, C., Della Bella, P., Giordano, U., Bloise, R., Giustetto, C., De Nardis, R., Grillo, M., Ronchetti, E., Faggiano, G., Nastoli, J., 2002. Natural history of Brugada syndrome: insights for risk stratification and management. Circulation 105, 1342-1347.
Schott, J. J., Alshinawi, C., Kyndt, F., Probst, V., Hoorntje, T. M., Hulsbeek, M., Wilde, A. A., Escande, D., Mannens, M. M., Le Marec, H., 1999. Cardiac conduction defects associated with mutations in SCN5A. Nature Genetics 23, 20-21.
Schulze-Bahr, E., Eckardt, L., Breithardt, G., Seidl, K., Wichter, T., Wolpert, C., Borggrefe, M., Haverkamp, W., 2003. Sodium channel gene (SCN5A) mutations in 44 index patients with Brugada syndrome: different incidences in familial and sporadic disease. Human Mutation 21, 651-652.
Shin, D. J., Jang, Y. S., Park, H. Y., Lee, J. E., Yang, K. J., Kim, E. M., Bae, Y. J., Kim, J. M., Kim, J. K., Kim, S. S., Lee, M. H., Chahine, M., Yoon, S J K., 2004. Genetic analysis of the cardiac sodium channel gene SCN5A in Koreans with Brugada syndrome. Journal of Human Genetics 49, 573-578.

Smits, J. P., Eckardt, L., Probst, V., Bezzina, C. R., Schott, J. J., Remme, C. A., Haverkamp, W., Breithardt, G., Escande, D., Schulze-Bahr, E., Le Marec, H., Wilde, A. A., 2002. Genotype-phenotype relationship in Brugada syndrome: electrocardiographic features differentiate SCN5A-related patients from non-SCN5A-related patients. Journal of the American College of Cardiology 40, 350-356.
Takahata, T., Yasui-Furukori, N., Sasaki, S., Igarashi, T., Okumura, K., Munakata, A., Tateishi, T., 2003. Nucleotide changes in the translated region of SCN5A from Japanese patients with Brugada syndrome and control subjects. Life Sciences 72, 2391-2399.
Todd, S. J., Campbell, M. J., Roden, D. M., Kannankeril, P. J., 2005. Novel Brugada SCN5A mutation causing sudden death in children. Heart Rhythm 2, 540-543.
Underhill, P. A., Jin, L., Lin, A. A., Mehdi, S. Q., Jenkins, T., Vollrath, D., Davis, R. W., Cavalli-Sforza, L. L., Oefner, P. J., 1997. Detection of numerous Y chromosome biallelic polymorphisms by denaturing high-performance liquid chromatography. Genome Research 7, 996-1005.
Viswanathan, P. C., Benson, D. W., Balser, J. R., 2003. A common SCN5A polymorphism modulates the biophysical effects of an SCN5A mutation. The Journal of Clinical Investigation 111, 341-346.
Wang, Q., Li, Z., Shen, J., Keating, M. T., 1996. Genomic organization of the human SCN5A gene encoding the cardiac sodium channel. Genomics 34, 9-16.
Wang, D. W., Makita, N., Kitabatake, A., Balser, J. R., George, A. L. Jr., 2000. Enhanced Na⁺ channel intermediate inactivation in Brugada syndrome. Circulation Research 87, E37-E43.
Wang, Q., Chen, S., Chen, Q., Wan, X., Shen, J., Hoeltge, G. A., Timur, A. A., Keating, M. T., Kirsch, G. E., 2004. The common SCN5A mutation R1193Q causes LQTS-type electrophysiological alterations of the cardiac sodium channel. Journal of Medical Genetics 41, e66.
Wilde, A. A., Antzelevitch, C., Borggrefe, M., Brugada, J., Brugada, R., Brugada, P., Corrado, D., Hauer, R. N., Kass, R. S., Nademanee, K., Priori, S. G., Towbin, J. A.; Study Group on the Molecular Basis of Arrhythmias of the European Society of Cardiology., 2002. Proposed diagnostic criteria for the Brugada syndrome: consensus report. Circulation 106, 2514-2519.
Yan, G. X., Antzelevitch, C., 1999. Cellular basis for the Brugada syndrome and other mechanisms of arrhythmogenesis associated with ST-segment elevation. Circulation 100, 1660-1666.
Ye, B., Valdivia, C. R., Ackerman, M. J., Makielski, J. C., 2003. A common human SCN5A polymorphism modifies expression of an arrhythmia causing mutation. Physiological Genomics 12, 187-193.

What is claimed is:

1. An isolated nucleic acid molecule encoding a mutant SCN5A protein corresponding to the wild type human SCN5A protein set forth in SEQ ID NO:2, wherein the mutant SCN5A protein has a W1191X mutation.

2. The isolated nucleic acid molecule according to claim 1, wherein the nucleic acid molecule has the sequence corresponding to that of the wild type human SCN5A cDNA set forth in SEQ ID NO:1 with a G to A substitution at nucleotide 3573.

3. A mutant SCN5A protein consisting of the amino acid sequence set forth in SEQ ID NO:2.

4. A method for detecting a cardiac disease or disorder associated with loss of cardiac sodium channel function in a subject, comprising:
(a) obtaining a biological sample from the subject; and
(b) detecting in said biological sample the presence or the expression of the nucleic acid molecule of claim 1 encoding the mutant SCN5A protein, wherein the detection of the presence or expression of the nucleic acid molecule encoding the mutant SCN5A protein is indicative of the cardiac disease or disorder associated with loss of cardiac sodium channel function.

5. The method according to claim 4, wherein the detecting is carried out by contacting the biological sample to an antibody specific to a mutant SCN5A protein consisting of the amino acid sequence set forth in SEQ ID NO:2.

6. The method according to claim 4, wherein the detecting is carried out by an amplification reaction, hybridization reaction or sequencing.

7. The method according to claim 4, wherein the cardiac disease or disorder is Brugada syndrome, long QT syndrome, atrial arrhythmia or progressive conduction disease.

8. The method according to claim 5, wherein the cardiac disease or disorder is Brugada syndrome.

9. A system for detecting a cardiac disease or disorder associated with loss of cardiac sodium channel function in a subject, comprising:
means for obtaining a biological sample from the subject; and
means for detecting in said biological sample the presence or the expression of the nucleic acid molecule of claim 1 encoding the mutant SCN5A protein, wherein the detection of the presence or expression of the nucleic acid molecule encoding the mutant SCN5A protein is indicative of the cardiac disease or disorder associated with loss of cardiac sodium channel function.
